(19) United States
(12) Patent Application Publication
(10) Pub. No.: US 2004/0028748 A1
(43) Pub. Date: Feb. 12, 2004

(54) Remedies for dermatophytosis

(76) Inventor: Koji Sasaya, Naka-ku, Yokohma-shi (JP)

Correspondence Address: J C Patents Inc, Suite 250, 4 Venture, Irvine, CA, 92618, US

(21) Appl. No.: 10/450,286

(22) PCT Filed: Dec. 3, 2001
(86) PCT No.: PCT/JP01/10552

(30) Foreign Application Priority Data
Dec. 11, 2000 (JP) 2000-376219

(51) Int. Cl. A61K 35/56; A61K 33/10

(52) U.S. Cl. 424/547; 424/687

(57) ABSTRACT

By obtaining a material usable in treating dermatophytosis from scallop shell, remedies for dermatophytosis which are harmless to the human body are prepared from natural materials while efficiently utilizing waste matters. Namely, remedies for dermatophytosis are obtained from a product formed by grinding shells having a crystalline structure due to the calcite structure of calcium carbonate.

### TECHNICAL FIELD

This invention relates to remedies for dermatophytosis.

### BACKGROUND ART

Various remedies for dermatophytosis have been provided for a long-term use for effectively preventing infection from other persons and to others persons. However, since these remedies contain a lot of chemically synthesized substances, adverse side effects to human bodies due to a long-term use thereof have been worried.

For solving the problem above, the inventors of this invention have found that a powder obtained by calcining and pulverizing scallop shells, which have been disposed of as wastes, can be utilized as a natural product having an anti-fungal activity without adverse effects on the human body. Accordingly, the object of this invention taking the above situation into consideration is to obtain a raw material for the remedies of dermatophytosis from the scallop shell, and to provide remedies of dermatophytosis that are harmless to the human body by taking advantage of natural products while attempting to effectively utilizing waste of the natural products.

### DISCLOSURE OF INVENTION

The invention is achieved by taking the above-mentioned problems into consideration to solve the problems by providing a remedy for dermatophytosis as a product obtained by pulverizing shells having a calcite crystal structure of calcium carbonate.

The above-mentioned product of this invention consists of the powdered shell having a calcite crystal structure of calcium carbonate, and it may be a mixture of calcium carbonate powder of porous particles and a calcium oxide powder prepared by calcining the calcium carbonate, and preferably it has good effect when it is dissolved in water.

### BEST MODE FOR CARRYING OUT THE INVENTION

The remedy for dermatophytosis of the present invention uses shells having at least a calcite crystal structure of calcium carbonate as a main ingredient thereof. Scallop shells are one of the representative examples of such shells, which are different from other shells in that most part of the scallop shells comprises this crystal structure. In the ecology of the scallops, they are featured in that they can quickly move for escaping from being captured by their predators (such as starfishes), as expressed as “swimming in the sea” by jetting sea water out of the shell by opening and shutting the shell. Shell adductors are well developed for enabling this action, and the shell itself is relatively thin and lightweight while having high mechanical strength. The shell comprises a calcite crystal structure of calcium carbonate aligned as a leaf-like texture at the inner face side of the shell. The calcite crystal structure of calcium carbonate has a plate-like texture in the inner layer of the shell (a core layer in the direction of thickness of the shell). Since the main part of the shell structure (the portion except the surface layer and hinge) is composed of the inner face side and inner layer of the shell, the shell can be thin and lightweight while having a high mechanical strength.

As described above with respect to the main part of the scallop shell, the inner face side has the leaf-like texture of the calcite crystal structure of calcium carbonate (needle-like crystals are aligned by being compacted as Kenzan (a needle-type frog), while the inner layer of the shell has a plate-like structure of the calcite crystal structure of calcium carbonate (many layers in which the needle-like crystals are aligned in one direction are stacked like a plywood, and the needle-like crystals are oriented in different directions for every layers). Accordingly, as described later, the powder obtained by pulverizing the scallop shells is porous and maintains calcite structure. On the other hand, while the calcium carbonate crystals are stacked by being spread on a plane in other kind of shells, and the shell has pearl luster, the granules prepared from such shells does not become porous.

As described above, the scallop shells are used as the raw material for obtaining the remedy of dermatophytosis, and though the shells have been disposed of as useless after removing the adductor of the shell, useful utilization of such waste is possible. The waste shells are collected, and dried in the sunlight for hardening. Then, the shells thus hardened by drying in the sunlight are pulverized to a granule of a diameter of about 200 μm. The pulverizing method is not particularly restricted, and pulverizers known in the art may be used. The porous granules of calcium carbonate are obtained as described above.

Subsequently, a part of the calcium carbonate powder comprising porous particles are used for obtaining a powder of calcium oxide by heating the particles at about 1000° C. for several minutes to several hours in a ceramic pot. A product is obtained by mixing the calcium oxide powder and calcium carbonate powder, and a remedy for dermatophytosis is prepared by dispersing the product in water.

Since the calcium carbonate powder in the remedy for dermatophytosis is the powder of scallop shell that is a natural product, as described above, it has an antimicrobial activity inherent to the natural products. The natural product is used herein as a contrast to chemically synthesized product, and cultivated scallop shells also belong to the natural product. The calcium oxide powder also exhibits a high antimicrobial activity due to its alkaline property. Accordingly, the remedy of the invention exhibits an antimicrobial activity inherent in the natural products and an antimicrobial activity due to alkaline property, and has properties that are evaded by microorganisms such as Tricophyton that causes dermatophytosis. Proliferation as well as survival of the microorganisms are inhibited at the lesion, and epidermis afflicted with dermatophytosis is restored by the human body's natural healing power.

The product is prepared by mixing the shell powder, which comprises porous particles and is the calcite crystal structure having the calcium carbonate powder, and the calcium oxide powder prepared by calcining the calcium carbonate powder, and by dispersing the mixture in water in the example above. However, the remedy of the invention is not limited thereto. For example, the product may comprise only the calcium carbonate powder or may comprise only the calcium oxide powder. Further, the product itself may be used without dispersing in water.

### Industrial Applicability

According to the present invention as described above, the remedy for dermatophytosis exhibits the antimicrobial activity inherent to the natural product and an alkaline property. Accordingly, the human body is not adversely affected by applying the remedy while inhibiting viability of microorganisms that is considered to cause the remedy of dermatophytosis to enable the natural healing power of the human body to be effectively manifested. It is another advantage that no adverse effect is observed after a long-term use since the product comprises natural materials without containing any chemically synthesized substances.

Use of the shell as the raw material permits the shell that has been mostly disposed of as wastes to be effectively used, thereby greatly contributing to reduce the amount of industrial wastes. The product of the invention can exert an excellent practical effect by providing the product at a low cost, since the remedy having a simple constitution is produced without using any complex process.

1. A remedy for dermatophytosis as a product obtained by pulverizing shells having a calcite crystal structure of calcium carbonate.

2. The remedy for dermatophytosis according to claim 1, wherein the product is a mixture of a powder of a shell, which has a calcite crystal structure of calcium carbonate and comprises porous particles, and a calcium oxide powder prepared by calcining the calcium carbonate powder.

3. The remedy for dermatophytosis as an aqueous dispersion of the product according to claim 1 or 2.
